(19) United States
(12) Patent Application Publication
(10) Pub. No.: US 2012/0172701 A1
(43) Pub. Date: Jul. 5, 2012

(54) Apparatus and Method for Acquiring Diagnostic Information

(75) Inventors: Ronaldus Frederik Johannes Holthuizen, Best (NL); Johan Michiel den Harder, Eindhoven (NL); Wendy de Kok, Hapert (NL)

(73) Assignee: Koninklijke Philips Electronics N.V., Eindhoven (NL)

(21) Appl. No.: 13/392,928

(22) PCT Filed: Sep. 16, 2010
(86) PCT No.: PCT/IB2010/054185

§ 371 Date: Feb. 28, 2012

(30) Foreign Application Priority Data
Sep. 22, 2009 (EP) 09170899.0

(51) Int. Cl. A61B 6/00

(52) U.S. Cl. 600/407

(57) ABSTRACT

Method and apparatus for acquiring diagnostic information comprising:-a data acquisition module for acquiring image data of at least a part of a person's anatomy,-a planning module defining with reference to a spatial position and orientation of an example anatomy at least one series of scanning steps to be executed with the data acquisition module on an actual anatomy,-a user interface to adjust imaging parameters to be used in a selected scanning step of a series of scanning steps, for acquiring image data of that actual anatomy, wherein-the user interface displays for every step of the selected series of scanning steps predefined scanning parameters pertaining to the example anatomy, and-the user interface is arranged to have a user select the actual imaging parameters to be used in each such step of the selected series of scanning steps with reference to a pre-established three-dimensional survey volume of the actual anatomy.

### FIELD OF THE INVENTION

The invention relates to an apparatus for acquiring diagnostic information comprising:
a data acquisition module for acquiring image data of at least a part of a person's anatomy,
a planning module defining with reference to a spatial position and orientation of an example anatomy at least one series of scanning steps to be executed with the data acquisition module on an actual anatomy,
a user interface to adjust imaging parameters to be used in a selected scanning step of a series of scanning steps, for acquiring image data of that actual anatomy.

### BACKGROUND OF THE INVENTION

Such an apparatus is known from WO2006/013499. In this known apparatus a fully automatic calculation and determination of the scanning parameters that will pertain to the imaging of a person's actual anatomy is carried out, which is based on default parameters that pertain to a scanning step concerning the anatomy. The parameters that are automatically calculated describe a set of slices to be acquired during the operation of the acquisition module on the actual anatomy. The user interface of the known apparatus is only used to allow the user—if desired—to modify the setting of the parameters that are automatically generated for the acquisition of the image data concerning the actual anatomy.

In this invention, similarly as in WO2006/013499, the data acquisition module can be any conceivable instrument, such as a magnetic resonance unit, a computer tomography unit, or any other type of instrument that is commonly used for the acquisition of image data of an anatomy. Although the invention is generally applicable for the acquisition of image data of any particular anatomy or part thereof, it is best explained with reference to the acquisition of cardiac image data when using a magnetic resonance unit. In view thereof in the following a discussion will be offered concerning the acquisition of cardiac image data with MRI, albeit that it is explicitly remarked that the invention is not restricted to this particular application.

Planning of cardiac examinations is perceived to be difficult for (novice) users in particular when using a magnetic resonance unit. In MRI considered planning of the slices to be imaged is required in order to combat the time that is needed for data acquisition. This remains a difficult task, also for more experienced users, due to the fact that this type of imaging does not occur frequently. There is therefore a need for an apparatus and method which offers a user-friendly possibility to plan the examination and the scanning steps to be carried out in the imaging of a particular anatomy.

### SUMMARY OF THE INVENTION

It is therefore an object of the invention to alleviate the planning task of users when preparing an acquisition of image data of a particular anatomy.

According to the invention an apparatus and a method is proposed in accordance with one or more of the appended claims. In accordance with the invention also a computer program is proposed to have the apparatus of the invention operate in accordance with the method of the invention.

In a first aspect of the invention the apparatus is

characterized in that
the user interface displays for every step of the selected series of scanning steps predefined scanning parameters pertaining to the example anatomy, and in that
the user interface is arranged to have a user select the actual imaging parameters to be used in each such step of the selected series of scanning steps with reference to a three-dimensional survey volume of the actual anatomy. This three dimensional survey volume may be acquired before hand, or it may be acquired in real-time at the time when the examination is contemplated. The survey volume may also be obtained from another source than the apparatus of the invention. Amongst the imaging parameters that can be adjusted are an off-center with reference to a center point of a pre-acquired volume, an angulation, size, and foldover direction taking into account knowledge of the shape of a human body, and the general location of a region of interest.

The user can then be guided through the scanning procedure and plan the scanning parameters for each scanning step that will apply. This obviates the need for a user to be knowledgeable on a high-level with respect to the anatomy to be examined, and secures that also a less experienced user is able to plan and implement an entire examination of an actual anatomy.

The object of the invention is best promoted when the user interface is arranged to have the user select the actual imaging parameters to be used in each step of the selected series of scanning steps, essentially in accordance with the predefined scanning parameters pertaining to the example anatomy.

Preferably the user interface comprises a visual display unit that during use provides a first, exemplary window showing an image of the example anatomy subjected to a selected scanning step, and a representation of the scanning parameters pertaining to the selected scanning step in relation to said example anatomy, and a second, working window showing an image of the actual anatomy, wherein the user interface is arranged to have the user establish with reference to the actual anatomy in said second, working window a representation of the actual scanning parameters that will pertain to the imaging of the actual anatomy in the planned scanning step.

The user can then simply compare the representation of the actual scanning parameters pertaining to the actual anatomy, with the representation of the exemplary scanning parameter as shown in the first, exemplary window, and decide whether the match between the two is sufficient for the purpose envisaged by the examination. This task can be further supported by arranging that the image of the actual anatomy shown in the second, working window corresponds in position and orientation to the example anatomy shown in the first, exemplary window.

One of the beauties of the invention is that for any given examination a complete series of scanning steps can be comprised in the planning module. At times however it may be preferable that each of the series of scanning steps in the planning module can be customized to meet site-specific preferences. In this way any particular examination institution, usually a hospital, can arrange to implement its own procedures.

It has proven to be very effective that the representation of the scanning parameters is shown as a line displayed on the visual display unit, which line represents a line of sight of the data acquisition module. This means that the user of the apparatus only has to manipulate a similar line in the second, working window in order to define the scanning parameters pertaining to the selected scanning step.

It is then further beneficial to have a computer-mouse as input device, and that the line to be displayed in the second, working window is drawable with a one click mouse operation.

### BRIEF DESCRIPTION OF THE DRAWINGS

The invention will hereinafter be further elucidated with reference to a drawing of an exemplary embodiment in accordance with the invention.

In the drawing:

FIG. 1 schematically shows the apparatus of the invention; and

FIG. 2 schematically shows images displayed during execution of the method of the invention on the visual display unit that forms part of the apparatus of the invention.

### DETAILED DESCRIPTION OF THE EMBODIMENTS

Wherever in the figures the same reference numerals are applied these numerals refer to the same parts.

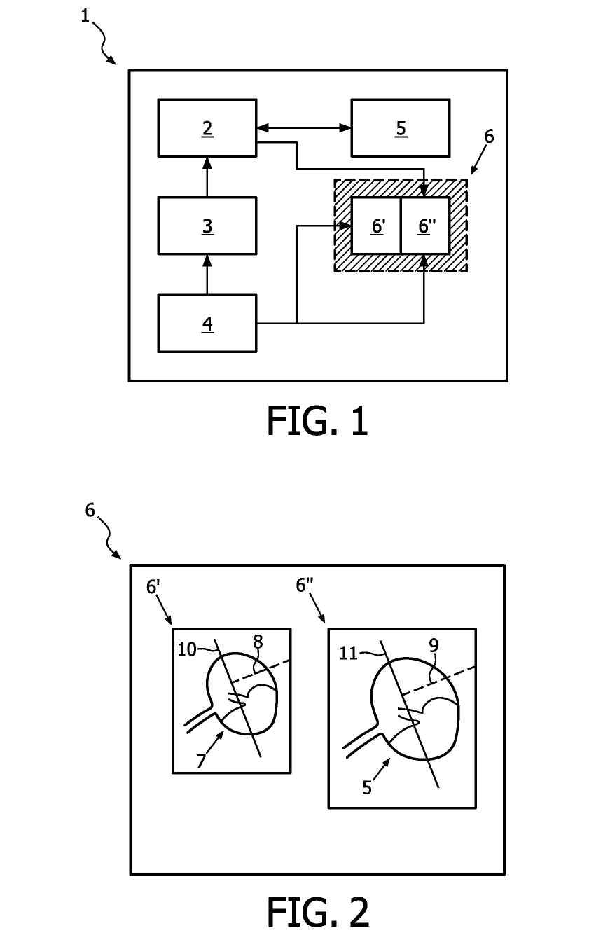

As mentioned in the introduction the data acquisition module to be used can be a magnetic resonance unit, a computer tomography unit, or any other type of commonly used data acquisition unit that is used for acquiring image data of a person's anatomy. In general such a data acquisition module is indicated with reference 2 in FIG. 1, and the anatomy to be examined is referred to with reference 5. The said data acquisition module 2 forms part of an apparatus 1 for acquiring diagnostic information pertaining to the anatomy 5, and is further provided with a planning module 3.

The planning module 3 has the purpose of defining with reference to a spatial position and orientation of an example anatomy a series of scanning steps to be performed when executing an examination, and which intends to have the data acquisition module 2 operate accordingly on the actual anatomy 5. When looking at the specific situation of having an MRI scanner perform a cardiac examination, this may for instance concern the object of acquiring an aortic valve view of the heart. A preferred planning path for the scanning operations to be executed for this purpose is to acquire subsequently the following views: transverse→right anterior oblique→nearly four chamber→short axis→four chamber→Basil short axis→left ventricular outflow tract→aortic valve. The user has to define the imaging parameters that are to be used in each of the scanning steps resulting in the just mentioned sequence of views, that collectively built the planning path that leads to the desired view of the aortic valve. It goes without saying that this is a complicated task if one cannot benefit from the apparatus and method according to the invention.

In the invention use is made of a user interface 4 to adjust the imaging parameters that are to be used in each of the scanning steps of the series of scanning steps, making up a complete examination card for acquiring the desired image data of the actual anatomy 5. For this purpose the user interface 4 displays for each single step in the selected series of scanning steps predefined scanning parameters pertaining to the example anatomy 7 in a window 6′ of a visual display unit 6. Further the user interface 4 is arranged to have a user select the imaging parameters that are to be actually used in the prevailing scanning step that is next to be undertaken with reference to the actual anatomy 5, a three-dimensional survey volume of which is shown in the window 6″ of the visual display unit 6. A preview showing an image visualizing the modified imaging parameters is shown in the window 6″ . This image can be a simulation or can originate from processing preacquired data, and/or a real-time imaging using the data acquisition module, or even a different data acquisition module.

The manner in which the user interface 4 preferably lets the user select the desired imaging parameters applicable to any prevailing scanning step that applies for the collection of a desired (intermediate) view, for instance for the purpose of going from a transverse view of the heart to the right anterior oblique view, can be explained best with reference to FIG. 2.

FIG. 2 shows the visual display unit 6 having a first, exemplary window 6′ showing an image 7 of an example anatomy which can be subjected to a scanning step to go from the shown transverse view of the heart to the right anterior oblique view. For this purpose it is required to acquire image data according to a line of sight represented by the striped line 8 with reference to the example anatomy 7. It may be helpful to also show a helpline 10 perpendicular to the line 8. This can promote the accuracy of positioning an actual line of sight with reference to an actual anatomy 5. Accordingly in a second, working window 6″ the visual display unit 6 shows the actual anatomy 5 and the scanning parameters represented by an adjustable line of sight embodied by the striped line 9 accompanied by a perpendicular helpline 11, the position and orientation of which lines the user can manipulate whilst having concurrently a view at the exemplary line of sight 8 and the perpendicular helpline 10 in the exemplary window 6′. In this manner the user is heavily assisted in drawing the line of sight 9 with reference to the actual anatomy 5 in order to realize an optimal match with the exemplary line of sight 8 in the exemplary window 6′, which will meet the intended purpose of acquiring the image data pertaining to the right anterior oblique view of the heart. This type of operation also allows the user to tune the said line of sight 9 representing the scanning parameters of the prevailing scanning step in order to address the peculiarities of the actual anatomy 5.

For the acquisition of the other or further views, respectively the acquisition of the eventual view of the aortic valve, the method and apparatus of the invention are to be operated similarly in the scanning steps that are to be executed to obtain such other or further views.

It will be appreciated to those skilled in the art that many variations are feasible within the scope of the invention as defined by the appended claims and without departing the scope of protection as afforded by these claims.

1. An apparatus (1) for acquiring diagnostic information comprising:
a data acquisition module (2) for acquiring image data of at least a part of a person's anatomy (5),
a planning module (2) defining with reference to a spatial position and orientation of an example anatomy (7) at least one series of scanning steps to be executed with the data acquisition module (2) on an actual anatomy (5),
a user interface (4) to adjust imaging parameters to be used in a selected scanning step of a series of scanning steps, for acquiring image data of that actual anatomy (5), characterized in that
the user interface (4) displays for every step of the selected series of scanning steps predefined scanning parameters (8) pertaining to the example anatomy (7), and in that
the user interface (4) is arranged to have a user select the actual imaging parameters (9) to be used in each such step of the selected series of scanning steps with reference to a three-dimensional survey volume of the actual anatomy (5).

2. An apparatus according to claim 1, characterized in that the user interface (4) is arranged to have the user select the actual imaging parameters (9) to be used in each step of the selected series of scanning steps, essentially in accordance with the predefined scanning parameters (8) pertaining to the example anatomy (7).

3. An apparatus according to claim 1, characterized in that the user interface (4) comprises a visual display unit (6) having during use a first, exemplary window (6′) showing an image of the example anatomy (7) to be subjected to a selected scanning step, and a representation (8) of the scanning parameters pertaining to the selected scanning step in relation to said example anatomy (7), and a second, working window (6″) showing an image of the actual anatomy (5), and that the user interface (4) is arranged to have the user establish with reference to the actual anatomy (5) in said second, working window (6″) a representation (9) of the actual scanning parameters that pertain to the imaging of the actual anatomy (5) in the selected scanning step.

4. An apparatus according to claim 3, characterized in that the image of the actual anatomy (5) shown in the second, working window (6″) corresponds in position and orientation to the example anatomy (7) shown in the first, exemplary window (6′).

5. An apparatus according to claim 1, characterized in that each of the series of scanning steps in the planning module (2) can be customized to meet site-specific preferences.

6. An apparatus according to claim 3, characterized in that the representation of the scanning parameters is indicated as a line (8, 9) displayed on the visual display unit (6), which line represents a line of sight of the data acquisition module (2).

7. An apparatus according to claim 6, having a mouse as input device, characterized in that the line (9) to be displayed in the second, working window (6″) is drawable with a one click mouse operation.

8. Method for acquiring diagnostic information comprising the steps:
acquiring image data Of at least a part of a person's anatomy (5),
planning with reference to a spatial position and orientation of an example anatomy (7) at least one series of scanning steps to be executed with a data acquisition module (2) on an actual anatomy (5),
adjusting imaging parameters to be used in a selected scanning step of a series of scanning steps, for acquiring image data of that actual anatomy (5),
characterized by the step of
displaying for every step of a selected series of scanning steps predefined scanning parameters (8) pertaining to the example anatomy (7), and by
arranging to have a user select the actual imaging parameters (9) to be used in each such step of the selected series of scanning steps with reference to a three-dimensional survey volume of the actual anatomy (5).

9. Method according to claim 8, characterized by the step to have the user select the actual imaging parameters (9) to be used in each step of the selected series of scanning steps, essentially in accordance with the predefined scanning parameters (8) pertaining to the example anatomy (7).

10. Method according to claim 8, characterized by showing in a first, exemplary window (6′) an image of the example anatomy (7) to be subjected to a selected scanning step, and a representation (9) of the scanning parameters pertaining to the selected scanning step in relation to said example anatomy (7), and by showing in a second, working window (6″) an image of the actual anatomy (5), whereby a user is arranged to establish with reference to the actual anatomy (5) in said second, working window (6″) a representation (9) of the actual scanning parameters that will pertain to the imaging of the actual anatomy (5) in the selected scanning step.

11. Method according to claim 10, characterized in that the image of the actual anatomy is shown in the second, working window (6″) with a position and orientation that corresponds to the example anatomy (5) shown in the first, exemplary window (6′).

12. Method according to claim 8, characterized in that the representation of the scanning parameters is shaped as a line (8, 9) with reference to the shown anatomy (7, 5), which line represents a line of sight of the data acquisition module (2).

13. A computer program comprising instructions for causing the apparatus according to claim 1 carry out his steps of the method.
